(19) United States
(12) Patent Application Publication
(10) Pub. No.: US 2021/0219980 A1
(43) Pub. Date: Jul. 22, 2021

(54) ENDOPROSTHESIS FOR A TOTAL VASCULAR EXCLUSION OF THE LIVER

(71) Applicant: Bruno Alberto Vittorio Truosolo, Rome (IT)

(72) Inventor: Bruno Alberto Vittorio Truosolo, Rome (IT)

(21) Appl. No.: 15/734,302

(22) PCT Filed: Jul. 5, 2018
(86) PCT No.: PCT/IT2018/000095

§ 371 Date: Dec. 2, 2020

(51) Int. Cl. A61B 17/12

(52) U.S. Cl. CPC A61B 17/12109; A61B 2017/00734; A61B 17/1204; A61B 2017/1205

(57) ABSTRACT

An endoprosthesis for a total vascular exclusion of the liver to be used in surgical operations. The endoprosthesis includes an endovenous catheter having the shape of a longitudinally extended cylinder, and being flexible in the transversal direction in order to be inserted preferably from the femoral vein or saphena vein, and directed to the inferior caval vein. The endovenous catheter also includes a self-expanding sheet that is rolled around itself and fixed at the distal part of the endovenous catheter. The sheet comprises a shape memory alloy having two states: a first state having a shape rolled around itself associated to a first temperature T₁, and a second state having an expanded shape associated to a second temperature T₂. The endoprosthesis includes means of heat generation and/or transmission between the endovenous catheter and the self-expanding sheet, and means of respective warming up or cooling down of the sheet.

The present invention regards a medical device that is represented by an improved endoprosthesis, that is a temporary venous transfemoral or trans-saphena device, for a total vascular exclusion of the liver.

This device results to be an improvement, in respect to the endoprosthesis for a total vascular exclusion of the liver described in the European patent EP 2851015, that has been granted already in favour of the same undersigned applicant.

As known, the endoprosthesis results to be particularly adapt to be used in most critical surgical operations involving the liver, like in example in major hepatectomy and in hepatic trauma with caval upper hepatic venous damage.

In fact, this device represents an internal and temporary endovenous bypass that permits the normal function of the caval vein to return the blood to heart, and at the same time permits, with a simultaneous Pringle maneuver, the total vascular exclusion of the hepatic parenchyma.
The liver receives blood coming from the portal vein and from the hepatic vein, and returns filtered blood to the caval system through the three upper hepatic veins.
The vascular exclusion of the liver, according to the European patent EP 2851015, consists in stopping the blood entering the liver, by blocking the hepatic peduncle (hepatic artery, common bile duct and portal vein—Pringle maneuver) with a clamp or a tourniquet, and stopping simultaneously the way out, so that the blood cannot go back to the liver from the inferior caval vein through the upper hepatic veins.
Therefore, it is used the Pringle maneuver, that is well known from the current surgical techniques, in order to stop the way of the blood going into the liver, and it is blocked the way out coming from the liver by closing the upper hepatic veins, by intervention directly on the caval vein, from the inside. This blocking of the way out is achieved by installation of an endoprosthesis, that is inserted directly from the femoral vein or saphena and then it is pushed inside the inferior caval vein, reaching with the distal part the caval lower and upper hepatic tract, blocking therefore with the lateral walls the holes connecting the upper hepatic veins to the caval vein. The endoprosthesis is composed of a main part having a cylindrical shape extending longitudinally. The part that, once it has been pushed in the caval vein, is placed close to the distal part of a inner guide, opens a spiral shaped self-expandable thin sheet, in order to bond steadily to the inside wall of the caval vein. The expansion saves an inner caval space that is necessary to keep the flow of the blood inside, so that the function of blood return to heart is saved.

With reference to the prior art, the endoprosthesis disclosed in the European patent EP 2851015 presents some drawbacks in the mechanism of expansion and rolling up of said sheet, when it is placed inside the caval vein.

In fact, said mechanism of expansion and following rolling up is achieved by using a releasing knob placed outside the main catheter, with a proper threading and counter threading in the main endovenous catheter.
A rotary motion in one sense, given to an inner guide inside the endoprosthesis at its proximal part, permits to retract the main venous catheter, so that the distal self-expandable sheet can be made free; the rotary motion of the knob in the opposite sense permits to recall the sheet, that has a proper squared shape able to facilitate its reinsertion in the main distal catheter end.
The transmission of the rotary motion all along the length of the endoprosthesis, in a clockwise or counter clockwise sense, from the proximal to the distal part, presents a series of drawbacks.

First of all, it is particularly complicated, from a technical point of view, to transmit a rotary motion at distance, in respect to the longitudinal axis, in a device that is placed following a tortuous arterious-venous conformation of a patient.

Then, the venous diameter changes along the endovenous path, therefore the insertion and installation of an inner device results to be very difficult. Furthermore, considering that the insertion of the endoprosthesis would be inside some very delicate and sensitive structures, it would be possible to cause very serious damages, inside the patient, that would result very hard to handle, making the surgical intervention very complex and characterized by a high level of risk for the patient.

Therefore, the main objective of the present invention is to propose an endoprosthesis for a total vascular exclusion of the liver, overcoming all the above drawbacks, permitting a comfortable use of the endoprosthesis because it can be applied and adapted to different patients having different size and extension of the inner anatomical structures.

Another objective is to keep the normal function of the caval vein to return the blood to heart, all during the time of a surgical intervention, avoiding a partial or total block of the blood circulation, and avoiding the need of a temporary extracorporeal circulation.

Another further objective is to achieve the expansion or the compression of the self-expandable sheet placed at the distal part, by using control means that include not only some mechanical and/or motion transmission instruments, but also some heat conducting means, so that the whole system could be particularly effective, comfortable in use, and could occupy a small space.

Therefore, it is specific subject of the present invention an improved endoprosthesis for a total vascular exclusion of the liver, to be used in most critical surgical operations like in example in major hepatectomy and in hepatic trauma with relevant venous damage, characterized in that comprising:

an endovenous catheter, having the shape of a cylinder extended longitudinally, and being flexible in the transversal direction in order to be inserted preferably from the femoral vein or saphena vein, directed to the inferior caval vein; said endovenous catheter having a diameter in the order of the inner diameter of the same femoral vein or saphena vein;

a self-expanding sheet, rolled around itself and fixed at the distal part of said endovenous catheter,

characterized in that:

said sheet is achieved by using a shape memory alloy, having two states: a first state having a shape rolled around itself, called martensite, associated to a first temperature T₁, and a second state having an expanded shape, called austenite, associated to a second temperature T₂,

said endoprosthesis comprises means of heat generation and/or transmission, between said endovenous catheter and said self-expanding sheet, and means of respective warming up or cooling down of said sheet; at said second temperature T₂, the self-expanding sheet changes its shape and achieves automatically the expanded shape, adapting itself perfectly to the surface of the caval vein, maintaining therefore the function of blood flowing back to the heart; at said first temperature T₁, the self-expanding sheet changes its shape and achieves automatically the shape rolled around itself, facilitating its reinsertion into the distal part of said endovenous catheter, so that, under control of an operator, the endovenous catheter is firstly installed by insertion from the femoral vein or saphena vein directed to the caval vein, with the self-expanding sheet placed in the caval tract of the upper hepatic veins; then, said mechanism of radial expansion of said self-expanding sheet is activated so that the lateral walls bond and close the holes connecting the upper hepatic veins to the inferior caval vein; therefore, the device permits the blood to flow inside the same self-expanding sheet preventing at the same time a return of blood to the liver; in such a way, with a simultaneous Pringle maneuver that stops the blood going to the liver, a total vascular exclusion of the liver is achieved.

As a memory shape material, it has been used preferably the NITINOL, that is a nichel-titanium alloy, commonly used in the biomedical field.

Some advantages of this material are the following:

the memory shape, that means the possibility to be object of a training, so that at a first temperature it changes its shape to a first predefined shape (martensite) and at a second temperature it changes its shape to a second predefined shape (austenite);

the pseusoelasticity, that permits to express a constant force under a large range of deformations, achieving respective intrinsically safe devices;

the biocompatibility, that under additional treatments makes the material extremely compatible with the human body, avoiding the release of substances and being resistant to corrosive agents;

the stress hysteresis, that in the self-expandable stents permits to express a soft force going from the stent to the blood vein, presenting instead a strong resistance to the deformation in front of external forces of compression.

The present invention will now be described for illustrative but not limitative purposes, according to its preferred embodiments, with particular reference to figures of the enclosed drawings, wherein:

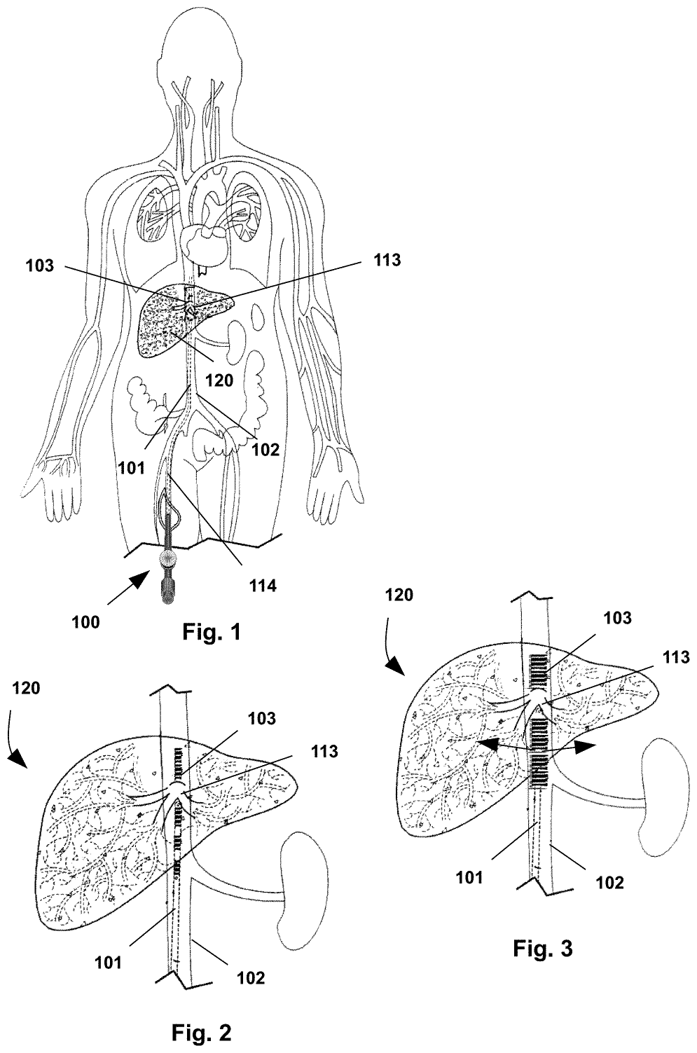

FIG. 1 is a front schematic view of a human body, wherein it is shown the insertion of the transfemoral endoprosthesis of the present invention;

FIG. 2 is a front schematic view of a particular of a liver of a human body, with the point of connection of the upper hepatic veins to the inferior caval vein, wherein it is shown the installation of the endoprosthesis having the radial surface in the compressed position;

FIG. 3 is a front schematic view of the same particular of a liver of FIG. 2, wherein it is shown the installation of the endoprosthesis having the radial surface in the expanded position, so that the holes connecting the upper hepatic veins to the inferior caval vein are closed, keeping at the same time the circulation of blood inside;

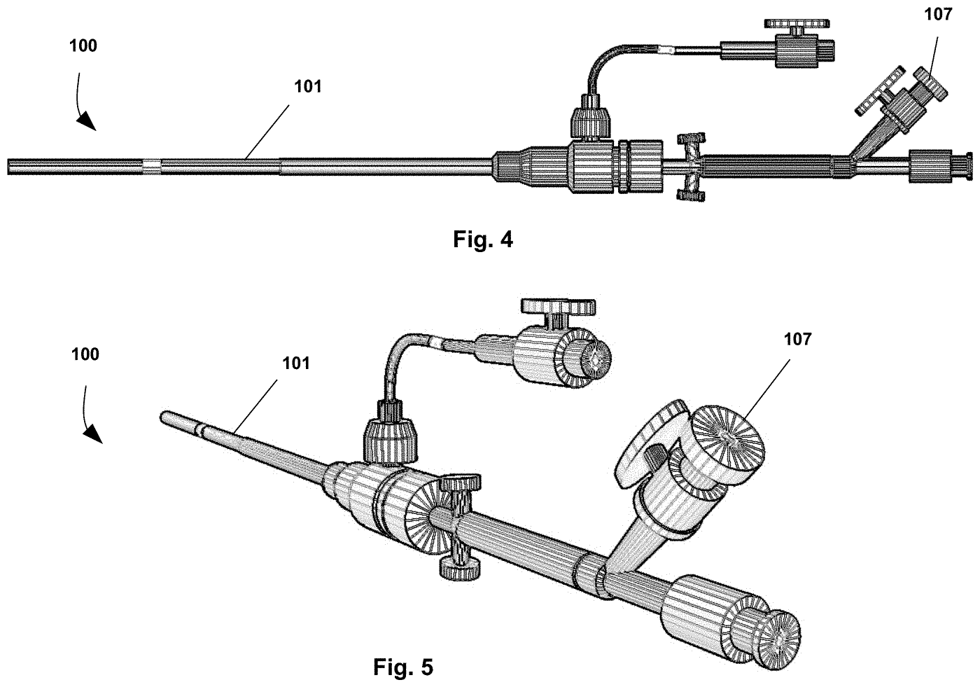

FIG. 4 is a lateral view of a temporary venous transfemoral or trans-saphena endoprosthesis for a total vascular exclusion of the liver;

FIG. 5 is a perspective lateral view of the same endoprosthesis of FIG. 4;

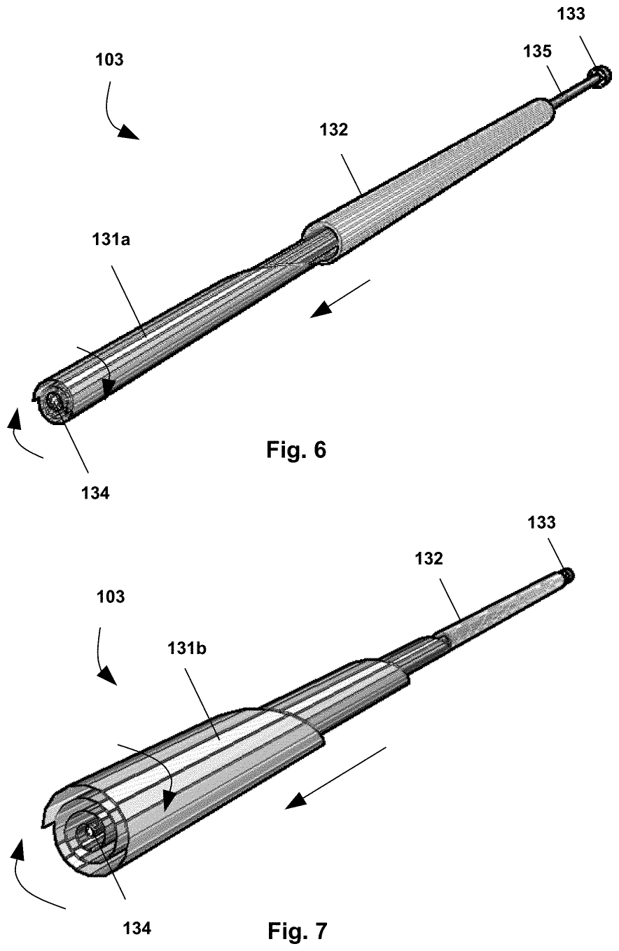

FIG. 6 is a perspective lateral view of the same endoprosthesis of FIGS. 4 and 5, where the self-expandable sheet is represented in a compressed shape;

FIG. 7 is a perspective lateral view of the same endoprosthesis of FIGS. 4 and 5, where the self-expandable sheet is represented in an expanded shape;

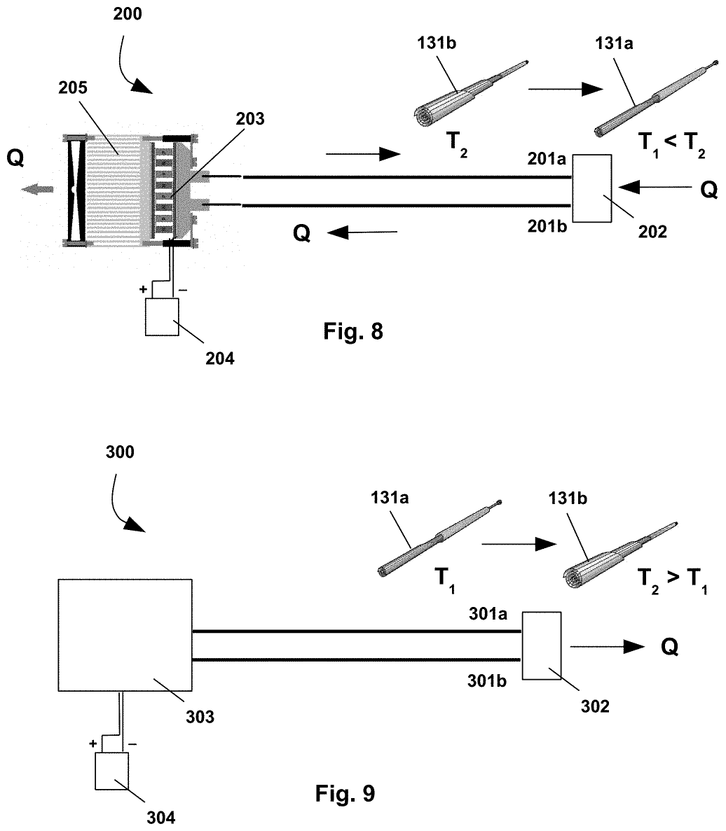

FIG. 8 is a schematic view of a cooling system for the self-expandable sheet, based on the Peltier effect, comprising a Peltier cells device and some thermally conductive means;

FIG. 9 is a schematic view of a warming system for the self-expandable sheet, based on the Joule effect, comprising an electric generator device and some thermally conductive means.

It is here underlined that only few of the many conceivable embodiments of the present invention are described, which are just some specific non-limiting examples, having the possibility to describe many other embodiments based on the disclosed technical solutions of the present invention.

FIGS. 4 and 5 show an improved endoprosthesis 100 for a total vascular exclusion of the liver 120 according to the present invention.

It can be used in most critical surgical operations like in example in major hepatectomy and in hepatic trauma with relevant venous damage.
The improved endoprosthesis 100 comprises essentially an endovenous catheter 101, having the shape of a cylinder extended longitudinally, and being flexible in the transversal direction in order to be inserted, as shown in FIG. 1, preferably from the femoral vein or saphena vein 114, directed to the inferior caval vein 102, and having a diameter in the order of the inner diameter of the same femoral vein or saphena vein 114 in a patient.
The endovenous catheter 101 further comprises a self-expanding sheet 103, rolled around itself and fixed at the distal part 133 of said endovenous catheter 101, as shown in FIGS. 6 and 7, and a mechanism of radial expansion of the same sheet 103, on command, from a first state having shape rolled around itself 131a to a second state having an expanded shape 131b.
In such a way, under control of an operator, the endovenous catheter 101 is firstly installed by insertion from the femoral vein or saphena vein 114 directed to the caval vein, with the self-expanding sheet 103 placed in the caval tract of the upper hepatic veins, as shown in FIG. 2. Then, said mechanism of radial expansion of said self-expanding sheet 103 is activated so that the lateral walls bond and close the holes connecting the upper hepatic veins 113 to the inferior caval vein 102, as shown in FIG. 3. Therefore, the device permits the blood to flow inside the same self-expanding sheet 103 preventing at the same time a return of blood to the liver 120. In such a way, with a simultaneous Pringle maneuver that stops the blood going to the liver 120, a total vascular exclusion of the liver 120 is achieved.
The above said sheet 103 is achieved by using a shape memory alloy, having two states: a first state having a shape rolled around itself 131a, called martensite, associated to a first temperature T₁, and a second state having an expanded shape 131b, called austenite, associated to a second temperature T₂.
The improved endoprosthesis 100 further comprises means of heat generation and/or transmission, between said endovenous catheter 101 and said self-expanding sheet 103, and means of respective warming up or cooling down of said sheet 103.
At said second temperature T₂, the self-expanding sheet 103 changes its shape and achieves automatically the expanded shape 131b, adapting itself perfectly to the surface of the caval vein, maintaining therefore the function of blood flowing back to the heart. At said first temperature T₁, the self-expanding sheet 103 changes its shape and achieves automatically the shape rolled around itself 131a, facilitating its reinsertion into the distal part 133 of said endovenous catheter 101.
FIGS. 6 and 7 show more in detail the mechanism of expansion as a spiral of the self-expandable sheet 103.
The self-expanding sheet 103 has a triangular or squared shape; it is connected and rolled up on a cylindrical container 132, represented i.e. by an elastic sheath made of a material having high thermal insulation, and it is placed at the distal part 133 of said endovenous catheter 101.
The cylindrical container 132 is connected to a supporting element 135, represented i.e. by flexible metallic wires, extending all along the length to the proximal part of said catheter 101 where it is connected to a control wheel of the translation. The supporting element 135, with the self-expanding sheet 103 at its end, is free to translate forward and backward in respect to the cylindrical container 132.
The endoprosthesis 100 includes a mechanism of forward or backward translation of said sheet 103 in respect to said cylindrical container 132, that is activated by said control wheel. A rotary motion in one sense—clockwise or counter clockwise—causes the sheet 103 to move forward outside said cylindrical container 132 with a progressive unrolling of the sheet 103 and therefore the expansion of said radial surface. After the rolling of the sheet 103 around itself, and therefore the compression of its radial surface, a rotary motion of the wheel in the opposite sense—counter clockwise or clockwise—causes the sheet 103 to move backward inside said cylindrical container 132.

The improved endoprosthesis 100 of the present invention includes various embodiments.

In a first embodiment, said temperature 12 is equal to the body temperature, therefore when the self-expandable sheet 103 is placed at the caval lower and upper hepatic tract, as shown in FIG. 2, and it is moved forward outside of container 132, then it starts to warm up reaching the body temperature and achieves automatically the expanded shape 131b (FIG. 7), adapting itself perfectly to the surface of the caval vein, maintaining therefore the function of blood flowing back to the heart.

After the surgical operation, in order to roll the self-expandable sheet 103 around itself, it is necessary to cool down the same sheet 103 to the temperature T₁. In fact, at temperature T₁ the sheet 103 changes its shape and achieves automatically the shape rolled around itself 131a (FIG. 6), facilitating its reinsertion into the distal part 133 of said endovenous catheter 101.
The cooling system for said self-expanding sheet 103 is achieved by embedding a cooling system 200 based on the Peltier effect.
In particular, as shown in FIG. 8, said means of heat generation and/or transmission comprise: a Peltier cells device 203 with its heat dissipation fins 205, power supplied by a battery 204; a thermally conductive element 201a, 201b placed along said endovenous catheter 101 that connects said Peltier cells device 203 to an heat exchanger 202; and an heat exchanger 202 placed in thermal contact with said self-expandable sheet 103.
In such a way, on operator's command, the cooling system 200 can be activated causing therefore a decrease of the temperature in the sheet 103, reaching the temperature T₁, and the sheet 103 changes its shape and achieves automatically the shape rolled around itself 131a, facilitating its reinsertion into the distal part 133 of said endovenous catheter 101.

In a second embodiment, said temperature T₁ is equal to the body temperature, therefore when the self-expandable sheet 103 is placed at the caval lower and upper hepatic tract, as shown in FIG. 2, and it is moved forward outside of container 132, then it is warmed up in order to achieve automatically the expanded shape 131b (FIG. 7), adapting itself perfectly to the surface of the caval vein, maintaining therefore the function of blood flowing back to the heart.

After the surgical operation, the sheet 103 starts to cool down reaching the body temperature T₁. In fact, at temperature T₁ the sheet 103 changes its shape and achieves automatically the shape rolled around itself 131a (FIG. 6), facilitating its reinsertion into the distal part 133 of said endovenous catheter 101.
The warming system for said self-expanding sheet 103 is achieved by embedding a warming system 300 based on the Joule effect.
In particular, as shown in FIG. 9, said means of heat generation and/or transmission comprise: an electric generator 303 power supplied by a battery 304; a thermally conductive element 301a, 301b placed along said endovenous catheter 101 that connects said electric generator 203 to an heat exchanger 302; and an heat exchanger 302 placed in thermal contact with said self-expandable sheet 103.
In such a way, on operator's command, the warming system 300 can be activated causing therefore an increase of the temperature in the sheet 103, reaching the temperature T₂, and the sheet 103 changes its shape and achieves automatically the expanded shape 131b, adapting itself perfectly to the surface of the caval vein, maintaining therefore the function of blood flowing back to the heart.

In a third embodiment, said first temperature T₁, where the self-expandable sheet 103 has a shape rolled around itself 131a, and said second temperature T₂, where the self-expandable sheet 103 has an expanded shape 131b, are absolutely independent in respect to the body temperature of the patient. Therefore, the warming or the cooling of the same sheet 103 are achieved, on operator's command, by activating respectively the above said warming system 300 or cooling system 200.

In this case, in order to protect the body of the patient from exposure to inner temperatures that could be potentially harmful, the present invention includes the possibility that all the components of the endoprosthesis 100, or part of them, are thermically insulated.

In particular, all the components of said endoprosthesis 100 that are inside the patient, including that achieving the transmission of cold or heat, are covered by a material having a high thermal insulation in respect to the outside, with particular reference to:
said endovenous catheter 101; said self-expanding sheet 103 and said means of heat generation and/or transmission.

In such a way, the decrease or increase of the temperature in the sheet 103, and the transmission of cold or heat inside the endovenous catheter 101, do not cause a decrease or increase of the local temperature, inside the anatomical structures of the patient, and therefore do not affect or interfere with the normal physiological functions of the same patient.

In order to insert and place the endovenous catheter 101 in its final position inside the patient, it is possible to use the images provided by a CT-scan (computed tomography).

According to an embodiment of the present invention, said self-expanding sheet 103 can include at its distal end a landmark composed of a radiopaque material. In such a way, the same landmark results to be visible on images coming from a CT-scan (computed tomography), and said endovenous catheter 101 can be guided in real time to its final position in the hepatic tract of the inferior caval vein.

As an alternative, the outer surface of said endovenous catheter 101 can present a millimetric scale continuously visible to the operator of intervention. In such a way, for a specific patient, starting from a CT-scan it is possible to detect the exact length of path that the endovenous catheter 101 should cover in order to reach the final position, and the millimetric scale permits the operator to understand exactly at any time where it is placed the endovenous catheter 101 in respect to the expected final position.

The self-expanding sheet 103 can be composed of a material that, in addition to be made of a shape memory alloy having two states, it is elastic and adapts itself perfectly to an irregular profile of the inner walls of the inferior caval vein 102, and to possible protrusions and concavities existing in the points of connection of the upper hepatic veins 113 in the same inferior caval vein 102.

The endovenous catheter 101 can be composed of modular and interchangeable parts having different sizes and extensions, with said self-expanding sheet 103 having different sizes and extensions, either in the initial position and in the expanded position. In such a way, the endoprosthesis 100 can be adapted and results to be as the most appropriate as possible, in respect to a specific circulatory system and anatomy of a specific patient, and can be used as an endovenous bypass in the most effective, appropriate and safe possible way.

With reference to the used materials, said self-expanding sheet 103 can be composed of a memory shape alloy, so called NITINOL, that is a nichel-titanium alloy, commonly used in the biomedical field, and it is covered by a material having a high waterproof and breathable feature, so called GORE-TEX, composed of stretched polytetrafluoroethylene.

Therefore, the above examples show that the present invention achieves all the proposed objectives. In particular, it permits to obtain an endoprosthesis for a total vascular exclusion of the liver, overcoming all the drawbacks of the prior art, permitting a comfortable use of the endoprosthesis, because it can be applied and adapted to different patients having different size and extension of the inner anatomical structures.

In particular, the endoprosthesis keeps the normal function of the caval vein to return the blood to heart, all during the time of a surgical intervention, avoiding a partial or total block of the blood circulation, and avoiding the need of a temporary extracorporeal circulation.

The present invention further achieves a fine adjustment of the expansion of the endoprosthesis, a better perception by the surgeon of its reached size, a better adhesion of it to the inner walls of the caval vein, and a more comfortable insertion and installation procedure.

Furthermore, the invention achieves the expansion or the compression of the self-expandable sheet placed at the distal part, by using control means that include not only some mechanical and/or motion transmission instruments, but also some heat conducting means, so that the whole system can be particularly effective, comfortable in use, and can occupy a small space.

The present invention has been described for illustrative but not limitative purposes, according to its preferred embodiments, but it is clear that modifications and/or changes can be introduced by those skilled in the art without departing from the relevant scope, as defined in the enclosed claims.

1. An endoprosthesis for a total vascular exclusion of the liver in surgical operations like major hepatectomy and in hepatic trauma with relevant venous damage, the endoprosthesis comprising:
an endovenous catheter having the shape of a cylinder extended longitudinally, and being flexible in the transversal direction in order to be inserted preferably from the femoral vein or saphena vein, and directed to the inferior caval vein; said endovenous catheter having a diameter in the order of the inner diameter of the same femoral vein or saphena vein;
a self-expanding sheet rolled around itself and fixed at the distal part of said endovenous catheter, said sheet comprising a shape memory alloy having two states: a first state having a shape rolled around itself called martensite, and associated to a first temperature T₁, and a second state having an expanded shape called austenite, and associated to a second temperature T₂, with T₂ being equal to the standard temperature of the human body and T₁<T₂;
said endoprosthesis comprises means of heat generation and/or transmission between said endovenous catheter and said self-expanding sheet, and means of respective warming up or cooling down of said sheet; at said second temperature T₂, the self-expanding sheet changes its shape and achieves automatically an expanded shape to match the surface of the caval vein, thereby maintaining the function of blood flowing back to the heart; at said first temperature T₁, the self-expanding sheet changes its shape and achieves automatically a shape rolled around itself to facilitate reinsertion into a distal part of said endovenous catheter, so that, under control of an operator, the endovenous catheter is firstly installed by insertion from the femoral vein or saphena vein directed to the caval vein, with the self-expanding sheet placed in the caval tract of the upper hepatic veins; then, said mechanism of radial expansion of said self-expanding sheet is activated so that the lateral walls bond and close the holes connecting the upper hepatic veins to the inferior caval vein; therefore, the device permits the blood to flow inside the same self-expanding sheet preventing at the same time a return of blood to the liver; in such a way, with a simultaneous Pringle maneuver that stops the blood going to the liver, a total vascular exclusion of the liver is achieved.

2. The endoprosthesis according to claim 1, wherein:
said self-expanding sheet has a triangular or squared shape and is connected and rolled up on a cylindrical container having an elastic sheath made of a material having high thermal insulation, and the self-expanding sheet is placed at the distal part of said endovenous catheter; said cylindrical container is connected to a supporting element having flexible metallic wires, extending all along the length to the proximal part of said catheter where it is connected to a control wheel of the translation; the supporting element, with the self-expanding sheet at its end, is free to translate forward and backward in respect to the cylindrical container;
said endoprosthesis includes a mechanism of forward or backward translation of said sheet in respect to said cylindrical container, that is activated by said control wheel: a rotary motion in one sense—clockwise or counter clockwise—causes the sheet to move forward outside said cylindrical container with a progressive unrolling of the sheet and therefore the expansion of said radial surface; after the rolling of the sheet around itself, and therefore the compression of its radial surface, a rotary motion of the wheel in the opposite sense—counter clockwise or clockwise—causes the sheet to move backward inside said cylindrical container.

3. The endoprosthesis according to claim 1, wherein:
said means of heat generation and/or transmission include a cooling system for said self-expanding sheet based on the Peltier effect, the cooling system comprising: a Peltier cells device having heat dissipation fins, a power supplied by a battery; a thermally conductive element placed along said endovenous catheter that connects said Peltier cells device to a heat exchanger placed in thermal contact with said self-expandable sheet,
so that the cooling system can be activated causing a decrease of the temperature in the sheet, reaching the temperature T₁, and the sheet changes its shape and achieves automatically the shape rolled around itself, facilitating its reinsertion into the distal part of said endovenous catheter.

4. The endoprosthesis according to claim 1, wherein:
said means of heat generation and/or transmission includes a warming system for said self-expanding sheet based on the Joule effect, in turn comprising: an electric generator power supplied by a battery; a thermally conductive element placed along said endovenous catheter that connects said electric generator to a heat exchanger placed in thermal contact with said self-expandable sheet,
so that the warming system can be activated causing an increase of the temperature in the sheet, reaching the temperature T₂, and the sheet changes its shape and achieves automatically the expanded shape, adapting itself perfectly to the surface of the caval vein, maintaining therefore the function of blood flowing back to the heart.

5. The endoprosthesis according to claim 1, wherein:
the components of said endoprosthesis that are inside the patient, including that achieving the transmission of cold or heat, are covered by a material having a high thermal insulation in respect to the outside, with particular reference to: said endovenous catheter; said self-expanding sheet and said means of heat generation and/or transmission,
so that, the decrease or increase of the temperature in the sheet, and the transmission of cold or heat inside the endovenous catheter, do not cause a decrease or increase of the local temperature, inside the anatomical structures of the patient, and therefore do not affect or interfere with the normal physiological functions of the same patient.

6. The endoprosthesis according to claim 1, wherein:
said self-expanding sheet includes a distal end having a landmark composed of a radiopaque material,
so that the same landmark results to be visible on images coming from a CT-scan, computed tomography, and said endovenous catheter can be guided in real time to its final position in the hepatic tract of the inferior caval vein.

7. The endoprosthesis according to claim 1, wherein:
the outer surface of said endovenous catheter presents a millimetric scale continuously visible to the operator of intervention,
so that, for a specific patient, starting from a CT-scan it is possible to detect the exact length of path that the endovenous catheter should cover in order to reach the final position, and the millimetric scale permits the operator to understand exactly at any time where it is placed the endovenous catheter in respect to the expected final position.

8. The endoprosthesis according to claim 1, wherein:
said self-expanding sheet is elastic and adapts itself perfectly to an irregular profile of the inner walls of the inferior caval vein, and to possible protrusions and concavities existing in the points of connection of the upper hepatic veins in the same inferior caval vein.

9. The endoprosthesis according to claim 1, wherein:
said endovenous catheter is composed of modular and interchangeable parts having different sizes and extensions, with said self-expanding sheet having different sizes and extensions, either in the initial position and in the expanded position,
so that the endoprosthesis can be adapted and results to be as the most appropriate as possible, in respect to a specific circulatory system and anatomy of the specific patient, and can be used as an endovenous bypass in the most effective, appropriate and safe possible way.

10. The endoprosthesis according to claim 1, wherein:
said self-expanding sheet is formed from a nickel titanium alloy, is covered by stretched polytetrafluoroethylene.
